(19) United States
(12) Patent Application Publication
(10) Pub. No.: US 2022/0054577 A1
(43) Pub. Date: Feb. 24, 2022

(54) MATERNAL BREAST MILK WITH NATURAL BIOACTIVE NUTRACEUTICAL EXTRACTS

(71) Applicant: Intermark Partners Strategic Management, LLC, Carmel, IN (US)

(72) Inventors: Glenn H. Sullivan, Carmel, IN (US); Guillermo E. Sanchez, Guatemala City (GT)

(21) Appl. No.: 17/405,332

(22) Filed: Aug. 18, 2021

Related U.S. Application Data
(60) Provisional application No. 63/067,031, filed on Aug. 18, 2020.

(51) Int. Cl. A61K 36/899

(52) U.S. Cl. CPC A61K 36/899

(57) ABSTRACT

Disclosed are nutraceutical compositions and formulations, and methods of using bioactive nutraceutical compositions and formulations of enzymatically hydrolyzed stabilized rice bran extract for the improvement of maternal breast milk, and for the mitigation of chronic malnutrition in infants, wherein the infant is breast feeding from a lactating mother ingesting said nutraceutical compositions or formulations in accordance with a prescribed regimen.

### CROSS-REFERENCE TO RELATED APPLICATIONS

This application claims priority to U.S. Application No. 63/067,031, filed on Aug. 18, 2020, which is incorporated herein by reference in its entirety.

### FEDERALLY SPONSORED RESEARCH OR DEVELOPMENT

[Not Applicable]

### TECHNICAL FIELD

The present invention relates to compositions comprising nutritionally enhanced derivatives (isolate) from Stabilized Rice Bran (SRB), and methods for the preparation and administration of said compositions to lactating mothers for the improvement of the quantity of breast milk and health and wellness of the mother. In certain aspects, the nutritionally enhanced derivatives (isolate) from SRB is administered before the onset of the breastfeeding period, or during the breastfeeding period, with concomitant improvements in the quantity of maternal milk.

### BACKGROUND

Chronic infant malnutrition (CIM) is a condition commonly associated with extreme poverty conditions and inadequate caloric-proteic intake by both mothers and infants during pregnancy, lactation and the transition into solid food intake.

CIM still affects about 165 million of children under five years of age in low- and middle-income countries around the world. The consequences of poor nutrition during this critical life stage are permanent intellectual impairment and physiological weakness, constituting in essence a sentence for a life of marginality, underperformance and food welfare.

There exist a need in the art to provide functional foods that provide sustainable nutrition to undernourished populations worldwide. Among other things, the present disclosure provides for the conversion of rice bran to its highest bioactive isolate derivatives in shelf-stable formulations, and for the nutraceutical formulations that mitigate malnutrition, including chronic infant malnutrition. The present disclosure further relates to the dietary supplementation, by lactating mothers, with stabilized rice bran enzymatic extracts (e.g., during the breastfeeding period) with an associated improvement in the quantity of maternal milk.

### SUMMARY OF THE INVENTION

The present disclosure provides compositions, and methods for preparing said compositions, comprising nutritionally enhanced derivatives (hydrolyzed isolate) from Stabilized Rice Bran (SRB). Also provided are methods and regimens for the administration of said compositions to lactating mothers for the improvement of the quantity of resultant breast milk. In certain aspects, the nutritionally enhanced derivatives (hydrolyzed isolate) from SRB is administered in accordance with a prescribed regimen during the breastfeeding period, with a concomitant improvement in the quantity of maternal milk and mother's nutritional wellness.

In a further aspect, the present disclosure relates to the dietary supplementation of lactating mothers with rice bran enzymatic extracts (e.g., before and/or during the breastfeeding period) with an associated improvement in the quantity of maternal milk.

In a still further aspect, the present disclosure provides evidence for the natural enzymatically enhanced rice bran extract having bioactive phytonutrients, that when consumed by breastfeeding mothers a few days before infant birth, increase the quantity of transitional milk and early mature milk to the level wherein the opportunity for infants to actually recover from the effects of low birth weight and realted chronic health conditions are enhanced. Low birth weight is a major cause of serious health complications among neonates.

In another aspect, the present disclosure provides enzymatically hydrolyzed and enhanced rice bran extracts, and regimens for its consumption by mothers during lactation, such that there is a remediation of intrauterine growth restriction (IUGR). What is more, there is a remediation of health problems observed during pregnancy, delivery, and/or after birth in children subject to IUGR, including low birth rate.

In yet another aspect, the present disclosure provides nutraceutical formulations in the form enzymatically hydrolyzed extracts (isolates) of stabilized rice bran having phytonutrients that function as one or more prebiotics for lactating mothers and their breastfed infants to achieve increased probiotic transfer to infants in the breastmilk.

In one aspect, nutraceutical formulations in accordance with the present disclosure enhance intestinal function of gut bacteria and overall nutritional efficacy for mitigating chronic health conditions in breastfed infants, and thereby increasing physical, cognitive, physiological, immunological, and behavior development.

In one aspect, the present disclosure provides for the consumption of nutraceutical formulations having enzymatically hydrolyzed phytonutrients in rice bran, which when formulated in accordance with the present disclosure, increase the post-partum breastmilk synthesis during at least the first six months of breastfeeding.

In a still further aspect, the physical, cognitive and physiological growth of infants breastfed throughout the first year of life by mothers having consumed bioactive nutrition during the exclusive breastfeeding and the transitional food period (e.g., second semester of life) continue to achieve enhanced growth and development well into their first year of life and beyond, thereby overcoming the health and wellness compromising effects associated with chronic malnutrition

### DETAILED DESCRIPTION

The Examples that follow are intended to be illustrative of the aspects and embodiments described above. Neither the above disclosure nor the Examples below should be viewed as limiting to the scope of the appended claims. One of skill in the art will appreciate that the disclosure is not limited by the particular terminology, which is used to describe and illustrate the various aspects of the disclosure.

To fully capitalize on the nutritional properties of a hydrolyzed concentrated rice bran enzymatic extract (RBEE) nutraceutical, with significantly increased bioactive capacity, protein, carbohydrate and antioxidant availability has been developed. The nutraceutical formulations disclosed herein resulted, in part, from the combination of hydrophilic (soluble fraction) and lipophilic (fiber fraction) fractions extracted from stabilized rice bran as described in U.S. Pat. No. 8,945,642, which is herein incorporated by reference in its entirety for all that it contains.

In one aspect, the methods disclosed herein use nutraceutical formulations comprising nutritionally enhanced isolates of stabilized rice bran having a soluble fraction/fiber fraction ratio of from 0.5 to 1.5; 0.75 to 1.25; 1.0 to 1.5; 1.0 to 1.4; 1.0 to 1.3, 1.0 to 1.20; or 1.0 to 1.15.

In a still further aspect, the nutraceutical formulations/compositions described herein comprise nutritionally enhanced isolates of stabilized rice bran having, in a 100 g sample: from 30 g to 50 g of a hydrophilic fraction and from 70 g to 50 g of a lipophilic fraction; from 35 g to 45 g of a hydrophilic fraction and from 65 g to 55 g of a lipophilic fraction; or from 40 g to 45 g of a hydrophilic fraction and from 60 g to 55 g of a lipophilic fraction. In one specific aspect, the nutraceutical formulations/compositions described herein comprise nutritionally enhanced isolates of stabilized rice bran having, in a 100 g sample, 42 g of a hydrophilic fraction and 58 g of a lipophilic fraction.

Alternatively, the nutraceutical formulations disclosed herein comprise a hydrophilic (soluble fraction)/lipophilic fractions (fiber fraction) blend comprising from 50% to 60%, from 55% to 60%, from 55% to 57%, or 57% of soluble hydrophilic fraction.

Still further, the nutraceutical formulations disclosed herein comprise a hydrophilic (soluble fraction)/lipophilic fractions (fiber fraction) blend comprising from 50% to 40%, from 45% to 40%, from 45% to 43%, or 43% lipophilic fiber fraction.

Blending and preparation of finished nutraceutical formulations is performed in accordance with specific ingredient proportions, such as that shown below in Table 1.

TABLE 1
Example of Base Ingredients in Lactating
Mothers/Infant Nutrition Formulation

| | 100 g of finished lactating |
|---|---|
| INGREDIENT | mothers product |
| Nutra-iso solubles (g) | 42.16 |
| Nutra-iso Fiberdex (g) | 48.51 |
| Micronutrient mix (g) | 0.27 |
| Sweetener | 9.06 |
| Total | 100 |

The customized micronutrient mix (i.e., premix) used to prepare the finished nutraceutical formula is a vitamin and mineral premix comprising, per 6.7 grams of the premix: 200 IU Vitamin D3 (as Cholecalciferol, USP-FCC); 30 IU Vitamin E (as acetate, USP-FCC); 90 mcg Biotin (USP); 6 mg Niacin (as Niacinamide, USP-FCC); 3 mg Pantothenic Acid (as D-Calcium Pantothenate, USP); 1.8 mcg Vitamin B12 (as Cyanocobalamin, USP); 60 mg Vitamin C (as Sodium Ascorbate, USP-FCC); 24 mcg Vitamin K1 (as Phytonadione, USP); 450 mg (Calcium (as Calcium Lactate, USP-FCC); 54 mcg Chromium (as Chromium Picolinate, USP); 37.5 mcg Iodine (as Potassium Iodide, USP-FCC); 120 mg Magnesium (as Magnesium Phosphate, USP-FCC); 22.5 mcg Molybdenum (as Sodium Molybdate); 350 mg Phosphorous (as Dipotassium Phosphate, anhy., FCC) and (Magnesium Phosphate, FCC); 1050 mg Potassium (as Dipotassium Phosphate, anhy., FCC) and (Potassium Chloride, USP-FCC); 17.5 mcg Selenium (as Sodium Selenite); and 6 mg Zinc (as Zinc Sulfate, USP-FCC).

In one example, micronutrients are added to the nutritionally enhanced isolate of stabilized rice bran in accordance with Table 2.

TABLE 2
Added Micronutrients

| | | | 40 grain ration | | |
|---|---|---|---|---|---|
| | | | | | % daily |
| | Natural | | | | requirement |
| | amount | | | | in lactating |
| | in 100 | | Original | | mothers |
| | Daily g | Daily | amount in | | fortified |
| Micro | Nutra- | Require- | Nutra- | | Nutra- |
| nutrient | Iso ™ | ment | Iso ™ | Added | iso ™ |

Vitamins

| | | | | | |
|---|---|---|---|---|---|
| Vitamin A | 0.00 | 1000.00 | 0.00 | 400.00 | 40.00 |
| (mcg) | | | | | |
| Vitamin D | 0.00 | 5.00 | 0.00 | 2.50 | 50.00 |
| (mcg) | | | | | |
| Vitamin C | 0.00 | 100.00 | 0.00 | 100.00 | 100.00 |
| (mc) | | | | | |
| B9-Folate | 31.38 | 500.00 | 12.67 | 487.33 | 100.00 |
| (mcg) | | | | | |
| B12 (mcg) | 0.00 | 2.80 | 0.00 | 2.80 | 100.00 |

Minerals

| | | | | | |
|---|---|---|---|---|---|
| Iron (mg) | 1.03 | 15.60 | 0.41 | 7.39 | 50.00 |
| Zinc (mg) | 0.98 | 22.60 | 0.39 | 11.69 | 53.47 |

| | |
|---|---|
| Total (g) | 0.120 |

The enhanced RBEE supplement of the present disclosure contains increased amounts of macronutrients and micronutrients, with increased bioavailability resulting from the dual (carbohydrases and proteases) enzymatic process applied to stabilized RB. The RBEE is obtained through a process by which a combination of carbohydrases and proteases, added to stabilized rice bran (SRB) in a multi stage process of centrifugation and dehydration, designed to extract and maximize the bioactive concentration of nutrients from the SRB's lipophilic and hydrophilic fractions. The nutritional composition/profile of one exemplar enhanced RBEE supplement in accordance with the present disclosure is shown in Table 3.

TABLE 3

| | Rice bran | | Per ration (40 g) | | |
|---|---|---|---|---|---|
| | enzymatic | | | | % |
| | extract typical | | | | Lactating |
| | analysis | | | | Women |
| Nutrient | 100 g | 40 g | Added | Total | RDA¹ |
| Energy | 448-468 | 180-188 | | 180-188 | 7 |
| (calories) | | | | | |
| Protein (g) | 12-15 | 4.8-6.0 | | 4.8-6.0 | 5.0 |
| Fat (g) | 27.90 | 11.16 | | 11.6 | 12.6 |
| Carbohydrates | 58.40 | 23.36 | | 23.36 | 6.4 |
| (g) | | | | | |
| Fiber (g) | 3.00 | 1.20 | | 1.20 | |
| Vitamin A | 106.70 | 42.68 | 400.00 | 442.68 | 44.3 |
| (mcg) | | | | | |
| Thiamin (mg) | 3.14 | 1.26 | | 1.26 | 96.6 |
| Riboflavin | 2.84 | 1.14 | | 1.14 | 71.0 |
| (mg) | | | | | |
| Niacin (mg) | 7.26 | 2.90 | | 2.90 | 17.1 |
| Pantothenic | 6.29 | 2.52 | | 2.52 | 35.9 |
| acid (mg) | | | | | |
| Pyridoxin (mg) | 3.51 | 1.40 | | 1.40 | 70.2 |
| Biotin (mcg) | 5.25 | 2.10 | | 2.10 | 6.0 |
| Folate (mcg) | 126.81 | 50.72 | 487.00 | 537.72 | 100.0 |
| Vitamin B12 | 0.64 | 0.26 | 2.80 | 3.06 | 100.0 |
| (mcg) | | | | | |
| Vitamin C | 10.62 | 4.25 | 100.00 | 104.25 | 100.0 |
| (mcg) | | | | | |
| Vitamin D | 1.91 | 0.76 | 2.5 | 3.26 | 65.3 |
| (mcg) | | | | | |
| Vitamin E (mg | 1.56 | 0.62 | | 0.62 | 3.3 |
| ET) | | | | | |
| Tocophenols | 2.11 | 0.84 | | 0.84 | |
| (mg) | | | | | |
| Tocotrienols | 2.28 | 0.91 | | 0.91 | |
| (mg) | | | | | |
| Phytosterols | 939.88 | 375.95 | | 375.95 | |
| (mg) | | | | | |
| Gamma- | 250.34 | 100.14 | | 100.14 | |
| oryzanol (mg) | | | | | |
| Ferulic acid | 3502.5 | 1401.00 | | 1401.00 | |
| (mcg) | | | | | |
| Vitamin K | 8.08 | 3.23 | | 3.23 | 5.6 |
| (mcg) | | | | | |
| Calcium (mg) | 145.7 | 58.28 | | 58.28 | 5.8 |
| Chromium | 283.91 | 113.56 | | 113.56 | 252.0 |
| (mcg) | | | | | |
| Copper (mcg) | 937.39 | 374.96 | | 374.96 | 72.0 |
| Iron (mg) | 4.14 | 1.66 | 7.39 | 9.05 | 58.0 |
| Magnesium | 542.3 | 216.92 | | 216.92 | 79.0 |
| (mg) | | | | | |
| Manganese | 12.95 | 5.18 | | 5.18 | 2.6 |
| (mg) | | | | | |
| Molybdenum | 35.85 | 14.34 | | 14.34 | 28.7 |
| (mcg) | | | | | |
| Phosphorus | 1366.0 | 546.40 | | 546.40 | 78.1 |
| (mg) | | | | | |
| Potassium (g) | 1.6 | 0.64 | | 0.64 | 12.5 |
| Zinc (mg) | 5.0 | 2.0 | 11.7 | 13.7 | 60.5 |

¹Required daily allowance as recommended by the U.S. Food and Nutrition Division of the National Institute of Medicine and the Nutrition Institute for Central America and Panama, INCAP.

Table 3. Lactating Mothers Formulation Nutritional Profile The nutraceutical compositions/formulations disclosed herein are prepared, in part, from the combination of hydrophilic (soluble fraction) and lipophilic (fiber fraction) fractions extracted from stabilized rice bran. Table 4 provides an example of a hydrophilic (soluble fraction) of a nutraceutical composition prepared and used in accordance with the present invention.

TABLE 4

| | Rice Bran Derivative, DLS 2412 | |
|---|---|---|
| Analysis | (per 100 g) | (per 10 g) |
| Moisture (g) | 2.25 | 0.23 |
| Ash (As ls) (g) | 3.34 | 0.334 |
| Protein N × 6.25 (As Is) (g) | 7.48 | 0.75 |
| Fat (Total) (As ls) (by GC) (g) | 25.287 | 2.529 |
| Fat (Saturated) (As Is) (by GC) (g) | 5.910 | 0.591 |
| Trans Fatty Acids (by GC) (g) | 0.011 | 0.001 |
| Fat (Polyunsaturated) (by GC) (g) | 9.279 | 0.928 |
| Fat (Monounsaturated) (by GC) (g) | 10.087 | 1.009 |
| Omega 3 Fatty Acids (g) | 0.335 | 0.034 |
| Omega 6 Fatty Acids (g) | 8.900 | 0.890 |
| Carbohydrates (g) | 61.64 | 6.16 |
| Calories | 501.96 | 50.20 |
| Calories from Fat | 227.58 | 22.76 |
| Total Dietary Fiber (As Is) (g) | 3.85 | 0.39 |
| Fiber, Soluble (As Is) (g) | 3.32 | 0.33 |
| Fiber, Insoluble (As ls) (g) | 0.53 | 0.05 |
| Total Sugar (g) | 19.20 | 1.92 |
| Cholesterol (mg) | 3.14 | 0.31 |
| Vitamin A (Iu) | ND | ND |
| Vitamin B1 (mg) | 2.46 | 0.25 |
| Vitamin B2 (mg) | 2.14 | 0.21 |
| Niacin (mg) | 7.57 | 0.76 |
| Pantothenic Acid (mg) | 8.91 | 0.89 |
| Vitamin B6 (mg) | 4.410 | 0.441 |
| Vitamin B 12 (mg) | ND | ND |
| Ascorbic Acid (mg) | ND | ND |
| Vitamin D (Iu) | ND | ND |
| Vitanlin E (Total) (Iu) | 2.56 | 0.26 |
| Vitamin K (mg) | ND | ND |
| Folic Acid (mg) | 0.150 | ND |
| Calcium (mg) | 19.50 | 1.95 |
| Chromium (mg) | 0.267 | 0.027 |
| Cobalt (mg) | ND | ND |
| Copper (mg) | 0.943 | 0.094 |
| Iron (mg) | 2.10 | 0.21 |
| Lead (mg) | ND | ND |
| Magnesium (mg) | 140.00 | 14.00 |
| Manganese (mg) | 3.80 | 0.380 |
| Molybdenum (mg) | 0.044 | 0.004 |
| Phosphorous (mg) | 560.00 | 56.00 |
| Potassium (mg) | 1100.00 | 110.00 |
| Sodium (mg) | 19.00 | 1.90 |
| Zinc (mg) | 1.750 | 0.175 |

Table 5 provides an example of a lipophilic (fiber fraction) of a nutraceutical composition prepared and used in accordance with the present invention.

TABLE 5

| | Rice Bran Derivative, DLS 5412 | |
|---|---|---|
| Analysis | (per 100 g) | (per 10 g) |
| Moisture (g) | 2.50 | 0.25 |
| Ash (As ls) (g) | 8.233 | 0.823 |
| Protein N × 6.25 (As Is) (g) | 15.14 | 1.51 |
| Fat (Total) (As ls) (by GC) (g) | 17.845 | 1.785 |
| Fat (Saturated) (As Is) (by GC) (g) | 3.908 | 0.391 |
| Trans Fatty Acids (by GC) (g) | 0.016 | 0.002 |
| Fat (Polyunsaturated) (by GC) (g) | 6.598 | 0.660 |
| Fat (Monounsaturated) (by GC) (g) | 7.323 | 0.732 |
| Omega 3 Fatty Acids (g) | 0.240 | 0.024 |
| Omega 6 Fatty Acids (g) | 6.326 | 0.633 |
| Carbohydrates (g) | 56.28 | 5.63 |
| Calories | 350.41 | 35.04 |
| Calories from Fat | 160.61 | 16.06 |
| Total Dietary Fiber (As Is) (g) | 33.84 | 3.38 |
| Fiber, Soluble (As Is) (g) | 9.87 | 0.99 |
| Fiber, Insoluble (As ls) (g) | 23.97 | 2.40 |
| Total Sugar (g) | 6.90 | 0.69 |
| Cholesterol (mg) | 3.07 | 0.31 |
| Vitamin A (Iu) | ND | ND |
| Vitamin B1 (mg) | 3.62 | 0.36 |
| Vitamin B2 (mg) | 3.34 | 0.33 |
| Niacin (mg) | 6.13 | 0.61 |
| Pantothenic Acid (mg) | 4.42 | 0.44 |
| Vitamin B6 (mg) | 2.860 | 0.286 |
| Vitamin B 12 (mg) | ND | ND |
| Ascorbic Acid (mg) | ND | ND |
| Vitamin D (Iu) | ND | ND |
| Vitanlin E (Total) (Iu) | 2.14 | 0.21 |
| Vitamin K (mg) | ND | ND |
| Folic Acid (mg) | 0.110 | 0.011 |
| Calcium (mg) | 54.00 | 5.40 |
| Chromium (mg) | 0.296 | 0.030 |
| Cobalt (mg) | ND | ND |
| Copper (mg) | 0.933 | 0.093 |
| Iron (mg) | 5.55 | 0.56 |
| Lead (mg) | ND | ND |
| Magnesium (mg) | 830 | 83 |
| Manganese (mg) | 19.500 | 1.950 |
| Molybdenum (mg) | 0.030 | 0.003 |
| Phosphorous (mg) | 1930 | 193 |
| Potassium (mg) | 1400.00 | 140.00 |
| Sodium (mg) | 21.75 | 2.18 |
| Zinc (mg) | 5.500 | 0.550 |

Compositions and formulations prepared and used in accordance with the present invention comprise antioxidants, which serve to protect the body from harmful free radicals that cause damage to cellular metabolism and lead to immune system impairment. In accordance with the present invention, compositions and formulations prepared and used herein comprise an antioxidant profile comprising at least the following: γ-Oryzanol (2200-3000 ppm) including Cycloatenyl ferulate, 24-Methylene cycloartanyl-ferulate, Campesteryl ferulate, β-Sitosteryl ferulate, and Stigmasteryl ferulate; Tocopherols/Tocotrienols (220-320 ppm) including α-Tocopherol, β-Tocopherol, γ-Tocopherol, S-Tocopherol, α-Tocotrienol, β-Tocotrienol, γ-Tocotrienol, and δ-Tocotrienol; Polyphenols including Ferulic acid, α-Lipoic acid, Methyl ferulate, ρ-Coumaric acid, ρ-Sinapic acid, Isovitexin, and Proanthocyanidins; Metal Chelators including Magnesium (6250-8440), Calcium (303-500), and Phosphorous (14700-17000); Phytosterols (2230-4400 ppm) including β-Sitosterol, Campesterol, Stigmasterol, Δ5-Avinsterol, Δ-Stigmastenol, Isofucosterol, Gramisterol, Citrostdienol, Obtusifoliol, Branosterol, 28-Homotyphasterol, 28-Homosteasteronic acid, 6-Deoxycastasterone, and β-Amyrin; Carotenoids (0.9-1.6 ppm) including α-Carotene, β-Carotene, Lycopene, Lutein, and Zeaxanthine; Amino Acids including Tryptophan (2100), Histidine (3800), Methionine (2500), Cystein (336-448), Cystine (336-448), and Argenine (10800); B-Vitamins including Thiamin (22-31), Riboflavin (2.2-3.5), Niacin (370-660), Pantothenic acid (36-50), Pyridoxin (29-42), Inositol/myoinositol (1200-1880), Biotin (0.1-0.22), Choline (930-1150), and Phytates (1500-1710); Polysaccharides including Cycloartenol ferulic acid-glycoside, Diferulic-acid complex, and Diferulic-acid+3 Glucose+2 calcium complex; Phospholipids including Phosphatidylcholine, Phosphatidylethanolamine, Lysophosphatidylcholine, and Lysophosphatidylethanolamine; Enzymes including Glutathione peroxidase, Methionine reductase, Superoxidase dismutase, Polyphenol oxidase, Aspartate amino transferase, Isozyme AAT-1, Isozyme AAT-2, and Coenzyme Q10.

In one aspect of the invention, administration of the compositions and formulations described herein results in an increase in the quantity of colostrum. Colostrum as used herein is defined as the initial fluid secreted by breasts during the first 4 days after delivery. In certain embodiments, mothers receiving compositions and formulations in accordance to a prescribed regimen have a from 5% to 10%, from 5% to 15%, from 5% to 20%, from 5% to 35%, from 10% to 35%, from 15% to 35%, from 20% to 35%, or from 15% to 25% increase in the volume of colostrum, compared to the average amount produced by a lactating mother not receiving said compositions and formulations. In certain embodiments, the volume of colostrum produced by lactating mothers receiving said compositions and formulations is between 2 ml to 20 ml, between 2 ml to 30 ml, between 10 ml to 20 ml, between 10 ml to 30 ml, or between 10 ml to 40 ml per breastfeed in the first three days.

In another aspect of the invention, administration of the compositions and formulations described herein results in an increase in the quantity of transitional milk. Transitional milk as used herein is as breastmilk synthesized between 5 days and 2 weeks after delivery. In certain embodiments, mothers receiving compositions and formulations in accordance to a prescribed regimen have a from 5% to 10%, from 5% to 15%, from 5% to 20%, from 5% to 35%, from 10% to 35%, from 15% to 35%, from 20% to 35%, or from 15% to 25% increase in the volume of transitional milk, compared to the average amount produced by a lactating mother not receiving said compositions and formulations. In certain embodiments, the volume of transitional milk produced by lactating mothers receiving said compositions and formulations is from 500 ml to 600 ml, 500 ml to 700 ml, from 500 ml 800 ml, from 500 ml to 900 ml, or from 500 ml to 1000 ml over a 24-hour period.

In another aspect of the invention, administration of the compositions and formulations described herein results in an increase in the quantity of early mature milk. Early mature milk as used herein is breastmilk produced between 2 and 4 weeks post delivery. In certain embodiments, mothers receiving compositions and formulations in accordance to a prescribed regimen have a from 5% to 10%, from 5% to 15%, from 5% to 20%, from 5% to 35%, from 10% to 35%, from 15% to 35%, from 20% to 35%, or from 15% to 25% increase in the volume of early mature milk, compared to the average amount produced by a lactating mother not receiving said compositions and formulations. In certain embodiments, the volume of early mature milk produced by lactating mothers receiving said compositions and formulations is from 500 ml to 600 ml, 500 ml to 700 ml, from 500 ml 800 ml, from 500 ml to 900 ml, or from 500 ml to 1000 ml over a 24-hour period.

In another aspect of the invention, administration of the compositions and formulations described herein results in an increase in the quantity of fully mature milk. Fully mature milk as used herein is breastmilk secreted 4 weeks after delivery and beyond. In certain embodiments, mothers receiving compositions and formulations in accordance to a prescribed regimen have a from 5% to 10%, from 5% to 15%, from 5% to 20%, from 5% to 35%, from 10% to 35%, from 15% to 35%, from 20% to 35%, or from 15% to 25% increase in the volume of fully mature milk, compared to the average amount produced by a lactating mother not receiving said compositions and formulations. In certain embodiments, the volume of fully mature milk produced by lactating mothers receiving said compositions and formulations is from 500 ml to 600 ml, 500 ml to 700 ml, from 500 ml 800 ml, from 500 ml to 900 ml, or from 500 ml to 1000 ml over a 24-hour period.

With regard to the prescribed regimen, mothers should start taking the compositions and formulations disclosed herein in the last 6 weeks, 5 weeks, or 4 weeks of pregnancy, and continue for at least 6 months, 7 months, 8 months, 9 months, 10 months, 11 months, or 12 months after birth of the child. In specific embodiments, the compositions and formulas described herein should be consumed/administered to mothers: starting within the last 4 weeks of pregnancy and continuing until at least 6 months after birth of the child; starting within the last 4 weeks of pregnancy and continuing until at least 12 months after birth of the child; starting within the last 6 weeks of pregnancy and continuing until at least 8 months after birth of the child; or starting within the last 8 weeks of pregnancy and continuing until at least 12 months after birth of the child.

The dose regimen includes the consumption/administration to mothers of between 20 g to 30 g per day of the compositions/formulations disclosed herein, of between 20 g to 35 g per day of the compositions/formulations disclosed herein, or between 20 g to 40 g per day of the compositions/formulations disclosed herein. The dosage form is preferably a powder to be mixed with water or milk for oral administration, with the addition of an optional sweetener.

In yet another aspect of the present invention, there are provided methods for the mitigation of negative health conditions in an infant, the method comprising the infant breast-feeding from a lactating mother ingesting, in accordance with a prescribed regimen, a nutraceutical composition or formulation disclosed herein. The health conditions to be mitigated in said breastfed infants include infant chronic malnutrition, growth retardation, low birth weight. The traits that are improved in said breastfed infants include, for example: physical; cognitive; motor skills; behavior; physiological; and/or immunological.

Physical traits that are improved in infants breast-feeding from a lactating mother ingesting the compositions and formulations described herein include: growth in length, weight, head circumference, and body-mass-index.

Cognitive traits that are improved in infants breast-feeding from a lactating mother ingesting the compositions and formulations described herein include improvement in the mental development index score, which evaluates abilities including: sensory/perceptual acuities, discriminations, and response; acquisition of object constancy; memory learning and problem solving; vocalization and beginning of verbal communication; basis of abstract thinking; habituation; mental mapping; complex language; and mathematical concept formation.

Motor skills and Behavior skills that are improved in infants breast-feeding from a lactating mother ingesting the compositions and formulations described herein include improvement in traits characterized Bayley Scales of Infant and Toddler Development tests. Specific motor skills improved include the degree of body control, large muscle coordination, finer manipulatory skills of the hands and fingers, dynamic movement, postural imitation, and the ability to recognize objects by sense of touch (stereognosis). Specific behavior skills that are improved include attention/arousal, orientation/engagement, emotional regulation. Also included are traits associated with Bayley's Social-Emotional scale, which regards ease of calming, social responsiveness, and imitation play. Also included are those traits associated with the Bayley's Adaptive Behavior scale, which regards the demands of daily life, including communication, self-control, following rules, and getting along with others.

1. A method for increasing the quantity of breast milk produced by a lactating mother, said method comprising the administration to said mother a composition comprising an enzymatically hydrolyzed rice bran extract having a hydrophilic soluble fraction/lipophilic fiber fraction blend, wherein said blend comprises per 100 g from 30 g to 50 g of a hydrophilic fraction and from 70 g to 50 g of a lipophilic fraction.

2. A method of mitigation chronic malnutrition in an infant, the method comprising the administration to said infant breast milk produced by a lactating mother consuming a composition comprising an enzymatically hydrolyzed rice bran extract having a hydrophilic soluble fraction/lipophilic fiber fraction blend, wherein said blend comprises, per 100 g, from 30 g to 50 g of a hydrophilic fraction and from 70 g to 50 g of a lipophilic fraction.

3. A composition for the dietary supplementation of lactating mother, said composition comprising an enzymatically hydrolyzed rice bran extract having a hydrophilic soluble fraction/lipophilic fiber fraction blend, wherein said blend comprises, per 100 g, from 30 g to 50 g of a hydrophilic fraction and from 70 g to 50 g of a lipophilic fraction.

4. A composition for the dietary supplementation that improves the health and wellness of the lactating mother.
